(19) United States
(12) Patent Application Publication
(10) Pub. No.: US 2004/0053415 A1
(43) Pub. Date: Mar. 18, 2004

(54) Ozone-in-water decay rate analyzer

(76) Inventor: Christopher R. Schulz, Aurora, CO (US)

Correspondence Address: Alfred J. Mangels, 4729 Cornell Road, Cincinnati, OH, 45241-2433, US

(21) Appl. No.: 10/244,147

(22) Filed: Sep. 14, 2002

(51) Int. Cl. G01N 35/08; G01N 33/00

(52) U.S. Cl. 436/135; 436/52; 436/53; 422/81; 422/82; 422/103

(57) ABSTRACT

Apparatus for determining the ozone decay rate constant for an ozone-in-water solution. The apparatus includes a single ozone residual sensor, to which a sample flow is directed to measure the initial ozone residual concentration, and to which a delayed flow stream of the same solution is directed to measure the ozone residual concentration after passage of a particular time interval. The time delay of the flow stream is achieved by passing the flow stream through a chamber that includes a tubing coil through which the sample stream flows, after which the ozone residual concentration of the delayed sample stream is measured by the same ozone residual sensor. A portion of the incoming flow stream is diverted to flow through the chamber to maintain the delayed sample stream at substantially the same temperature as the incoming flow stream.

### BACKGROUND OF THE INVENTION

1. Field of the Invention

The present invention relates to apparatus for determining the rate of decay of the ozone residual concentration in an ozone-in-water solution. More particularly, the present invention relates to a device for measuring an initial ozone residual concentration of an ozone-in-water solution, a time-delayed ozone residual concentration, and determining the first order ozone decay rate constant for a flow-through ozone contactor.

2. Description of the Related Art

The effectiveness of ozone disinfection in flow-through contactors depends on the ozone concentration of the water being treated and the time interval during which the water to be treated is exposed to ozone. A suitable way to accurately assess the effectiveness of ozone disinfection is to monitor the initial ozone residual concentration (“Cₒ”) of the ozonated solution, and the final ozone residual concentration (“C”) of the solution at the end of a contact time interval (“T”), to arrive at the factors that enable the calculation of the so-called CT product for the ozonated water.

The initial and final ozone residual concentrations can be measured directly by wet chemistry using indigo trisulphonate—a calorimetric reagent, or by using a commercially available ozone-in-water on-line analyzer. The ozone decay rate constant for the water being treated can then be calculated based upon first order decay kinetics using the equation:

K_{d}=ln(C/Cₒ)/T

where C is the final ozone residual in mg/L after a specified contact time, Cₒ is the initial ozone residual in mg/L at the start of a specified contact time, T is the contact time in minutes, and K_{d} is the decay rate constant in min⁻¹.

In operating ozone contactors the decay rate constant can be calculated by two methods. In the first method, a grab sample of ozonated water is collected in a beaker, the initial residual is measured using wet chemistry, the sample is held for a fixed time interval (say 1-5 minutes) at constant water temperature, the final residual is measured, and the first-order equation is used to compute the decay rate constant. In the second method, the ozone residual concentrations are measured at two fixed points along the ozone contactor using wet chemistry or two on-line ozone analyzers. A contact time value is assigned between the two sampling points and the first-order equation given above is utilized to compute the decay rate constant. The assigned contact time is based upon the measured flow rate through the contactor and the T₅₀ residence time distribution, which is determined from a tracer study of the contactor.

The first method has limited usefulness for disinfection compliance monitoring of ozone systems since it relies on grab sampling and wet chemistry analysis and cannot be automated. While the second method can be automated using two online ozone residual analyzers, the accuracy of the decay rate constant calculation is influenced by the magnitude and variability of flow short-circuiting through the contactor at different flow rates. Because flow short-circuiting results in changes in the residence time distribution at discrete sampling points along the contactor, it can result in uneven ozone residual concentrations across the width of the contactor at a given sample location. Consequently a single T₅₀ value determined from a tracer study may not be accurate where there are varying flow rates and varying hydraulic conditions in an operating ozone contactor.

It is an object of the present invention to overcome the problems and shortcomings noted above in connection with presently-utilized methods for determining the decay rate constant for an ozone-in-water solution.

### SUMMARY OF THE INVENTION

Briefly stated, in accordance with one aspect of the present invention, apparatus is provided for enabling the determination of an ozone decay rate constant for an ozone-containing solution. The apparatus includes a sensor for measuring an ozone residual concentration value of an ozone-containing solution. A first flow conduit conveys the ozone-containing solution to the sensor for measuring an initial ozone residual concentration value for the solution. A second flow conduit is provided for conveying a time-delayed flow component of the solution to the sensor for measuring a time-delayed ozone residual concentration value for the solution. The second flow conduit branches from the first flow conduit to divert a portion of the solution through a time delay chamber for delaying the diverted solution for a time interval before measuring the ozone residual concentration of the diverted solution.

### BRIEF DESCRIPTION OF THE DRAWINGS

The structure, operation, and advantages of the present invention will become further apparent upon consideration of the following description, taken in conjunction with the accompanying drawings in which:

FIG. 1 is a schematic diagram showing one embodiment of an ozone decay rate analyzer in accordance with the present invention in a first mode for measuring an initial ozone residual concentration;

FIG. 2 is a schematic diagram similar to that of FIG. 1 with the analyzer in a second mode for measuring a time-delayed ozone residual concentration;

FIG. 3 is an elevational view, in cross section, of a constant-temperature flow delay chamber for an ozone analyzer;

FIG. 4 is a cross-sectional view of the delay chamber shown in FIG. 3 taken along the line 4-4 thereof; and

FIG. 5 is a cross-sectional view of the delay chamber shown in FIG. 3 taken along the line 5-5 thereof.

### DESCRIPTION OF THE PREFERRED EMBODIMENTS

In the present invention there is provided apparatus for measuring the initial ozone residual concentration and for determining the first-order ozone decay rate constant for an ozone-in-water solution. The apparatus is suitable for use in flow-through ozone contactors. Precise contact times between two successive ozone residual measurements enable the accurate calculation of the ozone decay rate constant, thereby avoiding the inherent inaccuracies of the grab sample and twoanalyzer methods identified above. An analyzer in accordance with the present invention incorporates a single, standard, on-line ozone-in-water analyzer to provide an automatically operable, dual sampling arrangement for measuring ozone residual concentrations at the beginning (C₀) and at the end (C) of a variable contact time period (T).

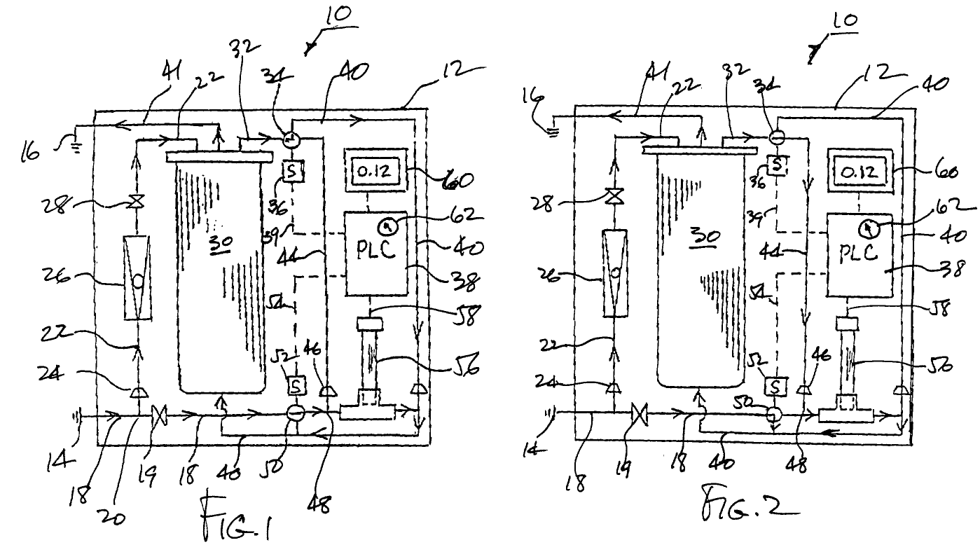

Referring now to the drawings, and particularly to FIGS. 1 and 2 thereof, there is shown in schematic form an embodiment of one form of ozone decay rate analyzer 10 in accordance with the present invention. Components of analyzer 10 can be enclosed within a suitable housing 12, which can be a NEMA 4X rated enclosure and can have dimensions of the order of about 16 inches in length, about 16 inches in width, and about 6 inches in depth. Housing 12 includes a sample inlet connection 14 and an outlet connection 16 for conveying the sampled ozone-in-water solution to a suitable disposal site, such as a drain, a sewer, or the like.

An inlet conduit 18 extends from inlet connection 14 to a tee 20. Branching from tee 20 is a first sample line branch 22 containing a reducer fitting 24 and including a flow measurement device 26, such as a rotameter, or the like. A flow control valve 28 is provided in branch 22 downstream of flow measurement device 26 to control the rate of flow of sample solution into a chamber 30 and into a coil of tubing (not shown) within the chamber. Inlet conduit 18 includes a flow control valve 19. Valves 28 and 19 are adjusted to balance the split of flows to the two branches, but only branch 22 is utilized to measure the flow for calculating the lag time.

An outlet conduit 32 extends from chamber 30 and is connected with the inlet of a first three-way valve 34 that can be operated by a solenoid 36 that is operatively connected with a programmable logic controller 38 by a conductor 39. One outlet of valve 34 communicates with a drain line 40 that extends to chamber 30 to conduct sample solution into the chamber to flow around the coil of tubing and to exit through outlet conduit 41 that communicates with outlet connection 16. The second outlet of three-way valve 34 communicates with a sample conduit 44 through a reducer fitting 46 to join at a tee 48 with inlet conduit 18 through a second three-way valve 50. Valve 50 controls flow through second branch 18. A solenoid 52 operates valve 50 and is operatively connected with programmable logic controller 38 by conductor 54. An outlet of tee 48 communicates with an ozone residual sensor 56 for sensing the ozone residual in the sample solution that passes through either of sample conduit 44 or conduit 18.

Ozone residual sensors suitable for measuring the ozone residual in an ozone-in-water solution can be obtained from sources such as Orbisphere, of Geneva, Switzerland; GLI International, Inc., of Loveland, Colo.; INUSA Corporation of Needham, Mass.; and Rosemount Analytical, of Irvine, Calif. Sensor 56 has an outlet that communicates with drain line 40 that extends to and communicates with the interior of chamber 30. A drain conduit 41 extends from chamber 30 to sample outlet 16.

Programmable logic controller 38 is operatively connected with solenoids 36, 52 of first and second three-way valves 34, 50, respectively, through respective conductors 39, 54 for transmitting electrical signals for controlling the operation of valves 34, 50. Controller 38 is also operatively connected with ozone residual sensor 56 through a conductor 58 for receiving ozone-in-water residual concentration values measured by that sensor. Additionally, controller 38 can be in the form of a microprocessor that is suitably programmed, and it includes, or is connected with, a display device 60 for displaying ozone residual concentration values and also for displaying the value of a calculated ozone decay rate constant.

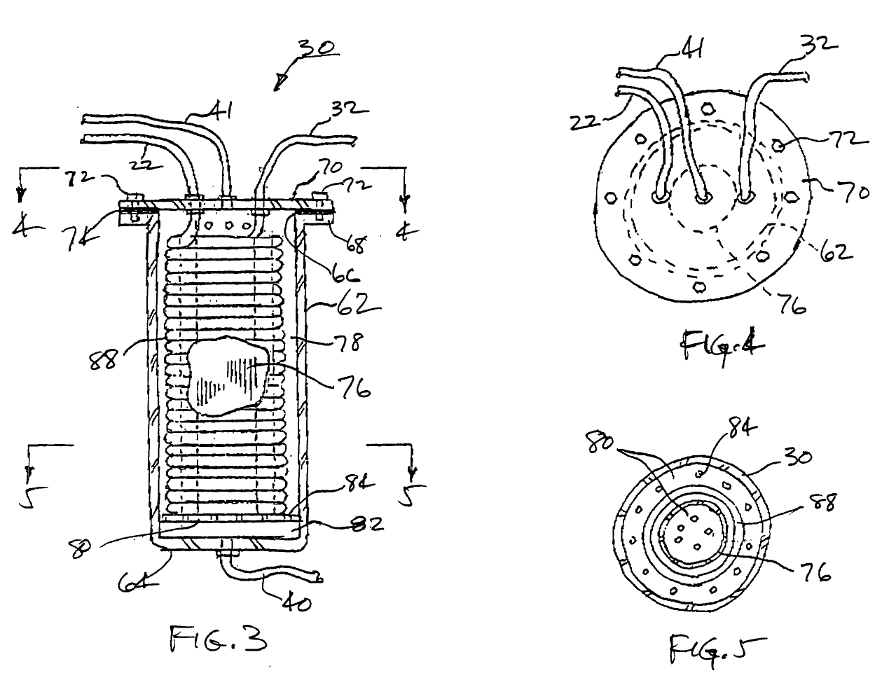

The structure of chamber 30 is shown in greater detail in FIGS. 3 through 5. Chamber 30 is a tubular component that includes a cylindrical sidewall 62, a closed bottom end 64, and an open top end 66. An annular flange 68 extends outwardly from side wall 62 at top end 66 of chamber 30, to which flange a top cover disc 70 is secured by a plurality of bolts 72 to completely close the interior of chamber 30. An annular gasket 74 is positioned between cover disc 70 and flange 68 to provide a liquid-tight seal therebetween.

A tubular core member 76 is substantially centrally positioned within chamber 30. Core member 76 has a smaller outer diameter than the inner diameter of chamber sidewall 62 to define therebetween an annular chamber 78. At the lower end of center core 76 is a flow distributor plate 80 that is spaced from closed bottom end 64 of tubular chamber 30 to define an inlet plenum chamber 82. Flow distributor plate 80 includes a plurality of spaced openings 84 to uniformly distribute a flow of drain solution that is introduced into chamber 30 through drain line 40. The drain solution flows into plenum chamber 82 and through openings 84 into core member 76 and also into annular chamber 78.

A sample-tubing coil 88 is carried within chamber 30. Coil 88 is in fluid communication with a sample inlet line 22 and a sample outlet line 32, each of which passes through cover disc 70. Coil 88 is wrapped around core member 76 and is completely immersed within the drain solution that flows into chamber 30 through inlet line 40 and that exits therefrom through outlet line 41. Coil 88 can be formed from any suitable material that is compatible with ozone, such as Teflon tubing. A coil formed from tubing having an inner diameter of ½ inch and an overall length of about 40 ft provides an effective flow delay time from entrance into the coil to exit from the coil of from about 1 to about 5 minutes at a sample solution flow rate range of from about 0.3 to about 1.5 lpm.

In operation, an ozone-in-water solution for which the ozone decay rate constant is to be determined is introduced into analyzer 10 at sample inlet connection 14. To measure the initial ozone residual concentration of the solution, three-way valve 50 is set to allow flow of the solution from inlet conduit 18, through tee 48, and directly to ozone residual sensor 56. The initial ozone-in-water residual concentration value Cₒ is measured by sensor 56, in mg/L, and that value is provided to programmable logic controller 40 through conductor 58, whereupon it can be displayed on display device 60. After passing sensor 56, the solution enters drain conduit 40 to flow to the inlet of chamber 30, around tubing coil 88, and then to exit from chamber 30 through drain line 41 to drain connection 16 for disposal.

While three-way valve 50 is in the position shown in FIG. 1 to allow sample solution to flow to sensor 56, three-way valve 34 is in the position to allow flow from chamber outlet conduit 32 into drain line 40, and to block flow into sample conduit 44. Thus, a portion of the incoming solution flows into branch line 22 and into and through tubing coil 88 within chamber 30. After exiting through outlet conduit 32, the solution enters drain line 40 to bypass sensor 56 and to flow into and through the interior of chamber 30 to drain conduit 41 and sample outlet 16.

In the flow configuration as represented in FIG. 1, the initial ozone residual concentration of the ozone-in-water solution is measured by sensor 56. To measure the ozone residual concentration in the ozone-in-water solution at a later time, to assess the ozone residual decay rate, each of three-way valves 34, 50 is moved to the positions represented in FIG. 2, to cause the time-delayed solution that has passed through tubing coil 88 within chamber 30 to flow to ozone residual sensor 56 through sample conduit 44.

Three-way valves 34, 50 can be operated by solenoids 36, 52, respectively, in response to signals from programmable logic controller 38 transmitted over conductors 39, 54, respectively. Thus, in the FIG. 2 flow configuration, the time-delayed sample solution flows to sensor 56 as the incoming sample solution is divided between inlet branch 22 and inlet conduit 18. That portion of the sample solution flowing through branch 22 is time-delayed within chamber 30, while the balance of the sample solution that flows through conduit 18 is diverted to drain line 40 by three-way valve 50.

The time-delayed, final ozone-in-water residual concentration value C is determined, in mg/L, and can be displayed in display 60. That value is provided to controller 38, which then calculates the decay rate constant by suitable logic and computation circuitry, for example by utilizing the first-order decay rate equation given above, based upon the initial ozone residual concentration value, the time-delayed ozone residual concentration value, and the delay time. That constant can then be utilized in other computations, such as calculating a predicted outlet ozone residual concentration value in an ozone-in-water disinfection process.

In carrying out the method described above, the user can provide as input data a desired sample flow rate, which can be of the order of from about 0.3 to about 2 lpm. A selector switch 62 can be provided on controller 38 to enable any of several analyzer and display options: first, the initial ozone residual concentration value; second, a time-delayed ozone residual concentration value; and third the calculated ozone decay rate constant value. Also provided as input quantities are the sample flow cycle time for the initial ozone residual concentration determination Cₒ, and for the time-delayed ozone residual concentration C, each of which times can be, for example, within the range of from about 1 to about 30 minutes, depending upon the sample flow rate through chamber 30.

In addition to calculating the decay rate constant, controller 38 can also serve to determine the time-delayed sample contact time. That determination can be made by providing within the controller a lookup table relating sample flow rate to contact time for a particular configuration.

As will be apparent, chamber 30 serves to impose on the incoming sample stream entering analyzer 10 at inlet 14 a time delay before measurement of a second ozone residual concentration. The interval between an initial sample ozone residual concentration value and a time-delayed sample ozone residual concentration value permits the determination of the decrease over time of the ozone residual concentration in the sample stream. The time delay is a function of the sample stream flow rate, and that flow rate can be controlled by the operation of flow control valves 19 and 28. Additionally, chamber 30 serves to maintain the time-delayed sample solution at the same temperature as that of the incoming sample solution, to permit a more accurate decay rate constant determination that is substantially independent of temperature changes that could otherwise occur in the sample solution between the respective ozone residual concentration determinations.

Although particular embodiments of the present invention have been illustrated and described, it will be apparent to those skilled in the art that changes and modifications can be made without departing from the spirit of the present invention. Accordingly, it is intended to encompass within the appended claims all such changes and modifications that fall with the scope of the present invention.

What is claimed is:

1. Apparatus for enabling the determination of an ozone decay rate constant for an ozone-containing solution, said apparatus comprising:
a. a sensor for measuring an ozone residual concentration value of an ozone-containing solution;
b. a first flow conduit for conveying the ozone-containing solution to the sensor for measuring an initial ozone residual concentration value for the solution;
c. a second flow conduit for conveying a time-delayed flow component of the solution to the sensor for measuring a time-delayed ozone residual concentration value for the solution, wherein the second flow conduit branches from the first flow conduit to divert a portion of the solution for delaying the diverted portion of the solution for a time interval before measurement of the ozone residual concentration of the diverted portion of the solution.

2. Apparatus in accordance with claim 1, including a predetermined length of conduit connected with the second flow conduit and through which the diverted portion of the solution passes to time delay the diverted portion of the solution before the ozone residual concentration of the diverted portion of the solution is determined.

3. Apparatus in accordance with claim 2, wherein the predetermined length of conduit is in the form of a coil.

4. Apparatus in accordance with claim 3, wherein the predetermined length of conduit is positioned within a time delay chamber through which the diverted portion of the solution passes.

5. Apparatus in accordance with claim 4, wherein the-time delay chamber is a closed vessel and includes an inlet and an outlet for conveying ozone-containing solution through the chamber and around the coil to maintain the diverted portion of the solution at substantially the same temperature as that of the ozone-containing solution that passes through the first flow conduit.

6. Apparatus in accordance with claim 1, including a processor operatively connected with the sensor for receiving from the sensor signals representative of ozone residual concentration values.

7. Apparatus in accordance with claim 6, wherein the processor is a microprocessor.

8. Apparatus in accordance with claim 6, wherein the processor is programmed to calculate the ozone decay rate constant for the solution by use of the following first-order decay rate equation:
K_{d}=ln(C/Cₒ)/T
wherein C is a final ozone residual in mg/L after a delay time, Cₒ is an initial ozone residual in mg/L at the start of a specified contact time, T is the contact time in minutes, and K_{d} is the decay rate constant in min⁻¹.

9. Apparatus in accordance with claim 8, including a display for displaying at least one of a measured initial ozone residual concentration for the solution, a measured time-delayed ozone residual concentration for the diverted portion of the solution, and a calculated ozone decay rate constant.

10. Apparatus in accordance with claim 1, including a flow meter and a flow control valve positioned in the second flow conduit for controlling the rate of flow of solution through the second flow conduit.

11. Apparatus in accordance with claim 1, including a respective valve positioned within each of the first and second flow conduits for selectively controlling flow of solution from the first and second flow conduits to the sensor and for allowing flow of solution to the sensor from only one of the first and second flow conduits at a given time.

12. Apparatus in accordance with claim 11, wherein the valves are three-way valves.

13. Apparatus in accordance with claim 12, wherein the valves are solenoid-operated valves.

14. Apparatus in accordance with claim 6, including respective solenoid-operated three-way valves positioned within each of the first and second flow conduits for selectively controlling flow of solution from the first and second flow conduits to the sensor and for allowing flow of solution to the sensor from only one of the first and second flow conduits at a given time, and wherein the solenoid-operated valves are operatively connected with the processor.

15. Apparatus in accordance with claim 14, including drain conduits extending from outlets of each of the solenoid-operated valves to divert to a drain conduit solution that is not conveyed to the sensor.

16. Apparatus in accordance with claim 15, wherein the drain conduits communicate with a time-delay chamber positioned in the second flow conduit and having an interiorly-positioned length of conduit for time delaying a portion of solution, and wherein flo′N from the drain conduits passes around the interiorly-positioned length of conduit to maintain flow that passes therethrough at a temperature substantially equal to a temperature of solution that flows through the first flow conduit.
